(19) United States
(12) Patent Application Publication
(10) Pub. No.: US 2008/0294204 A1
(43) Pub. Date: Nov. 27, 2008

(54) SYSTEMS, METHODS, AND DEVICES FOR SOFT TISSUE ATTACHMENT TO BONE

(75) Inventors: Paul E. Chirico, Campbell, CA (US); Benny M. Chan, Fremont, CA (US)

Correspondence Address: SHAY GLENN LLP, 2755 CAMPUS DRIVE, SUITE 210, SAN MATEO, CA, 94403, US

(73) Assignee: SPINEWORKS MEDICAL, INC., San Jose, CA (US)

(21) Appl. No.: 12/044,880

(22) Filed: Mar. 7, 2008

Related U.S. Application Data
(60) Provisional application No. 60/905,508, filed on Mar. 7, 2007.

(51) Int. Cl. A61B 17/84

(52) U.S. Cl. 606/327

(57) ABSTRACT

The soft tissue anchor devices, systems and methods described herein may aid in the attachment of ligaments, muscles, tendons, and other soft tissues to bone. These soft tissue anchor devices typically include a self-expanding bone anchor region including an elongate shaft that may be positioned within bone and expanded to anchor to the bone, and one or more soft tissue attachment regions adapted to attach soft tissue thereto.

### CROSS REFERENCE TO RELATED APPLICATIONS

This application claims priority to U.S. Provisional Patent Application Ser. No. 60/905,508, titled “SOFT TISSUE ATTACHMENT TO BONE ANCHOR”, filed Mar. 7, 2007.

This application is related to U.S. patent application Ser. No. 11/468,759, titled “Implantable Devices and Methods for Treating Micro-Architecture Deterioration of Bone Tissue”, filed Aug. 30, 2006; U.S. patent application Ser. No. 12/025,537, titled “Methods and Devices for Stabilizing Bone Compatible for use with Bone Screws”, filed Feb. 4, 2008; U.S. patent application Ser. No. 12/024,938, titled “Systems, Devices and Methods for Stabilizing Bone”, filed Feb. 1, 2008; and U.S. patent application Ser. No. 12/041,607, titled “Fracture Fixation System and Method”, filed Mar. 3, 2008. All of these applications are incorporated herein by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are systems, devices, and methods for attaching soft tissue to bone. In particular, described herein are systems, devices, and methods for attaching muscles, tendons, and ligaments to bone.

### BACKGROUND OF THE INVENTION

Soft tissues, including ligaments and tendons, may normally attach to bone. For example, ligaments are the fibrous tissues that connect bone to bone, and tendons are the fibrous tissues that connect muscle to bone. Soft tissues such as tendons and ligaments may be injured by excessive stretching, which can result in a partial or full tear of the ligament from the bone. Partial or complete separation of soft tissue from bone may be part of the pathology of ankle sprains and sprains of the knee, such as rupture of the anterior cruciate ligament (ACL). Tendon ruptures occur less frequently than sprains but commonly occur in the quadriceps, the rotator cuff, the biceps, and the Achilles tendon.

Full tears of ligaments or tendons often require surgery to repair. Reattachment of the soft tissue to bone is typically achieved by using anchoring devices such as staples, tacks, suture anchors, screws, pins, or dowels. In some situations, such as with ACL reconstruction, the damaged ligament can be replaced with other ligaments from the patient's body or with cadaveric ligaments. A graft tendon can include pieces of bone at its extremities to assist in attachment of the tendon to bone. Ligament and tendon surgeries are often invasive procedures resulting in scarring, discomfort, and a lengthy rehabilitation period. Additionally, soft tissue attachment to bone devices without sufficient strength to hold the soft tissue in place can fail, requiring additional surgery to reattach the soft tissue.

Thus, substantial challenges remain in designing soft tissue attachment to bone devices. In particular, devices that are capable of securely fastening soft tissue to bone in a minimally invasive manner would be beneficial. Described herein are devices, systems and methods that may address these and other challenges.

### SUMMARY OF THE INVENTION

Described herein are soft tissue anchoring devices, systems for anchoring soft tissue, and methods of anchoring soft tissue. In general, a soft tissue anchor device may include a bone anchor region that connects to the bone, and a soft tissue attachment region. The bone anchor region is typically a self-expanding bone anchor comprising multiple struts that are pre-biased in an anchoring, or expanded, configuration. The soft tissue attachment region may include a soft tissue holdfast, with which tissue can be secured. These soft tissue anchoring devices may be one-part, in which the bone anchor region and the soft tissue attachment site are integrally connected, or they may be two-part, in which the bone anchor region is connectable to the soft tissue attachment region. A connector region may be included between the bone anchor region and the soft tissue attachment region. An applicator may be used with the soft tissue anchors described herein.

For example, described herein are soft tissue anchor devices comprising a distal bone anchor region having an elongate shaft including a plurality of self-expanding struts extendable therefrom, the bone anchor region adapted to be positioned within a bone and having an expanded deployed profile and a collapsed delivery profile, and a proximal a soft tissue holdfast configured to fixedly secure a soft tissue.

As mentioned, the devices may also include a connecting region between the elongate shaft and the tissue attachment region. For example, the connecting region may be a flexible connecting region (e.g., a tether, a chain, etc.). The connecting region may be an adjustable connecting region. For example, the connecting region may be shortened or lengthened. In some variations the connecting region is a connector that permanently or temporarily connects the two adjacent regions (the bone anchor region and the soft tissue attachment regions).

The bone anchor region may be adapted to cut through the bone during expansion from the collapsed delivery profile to the expanded deployed profile. For example, each self-expanding strut can include a cutting surface adapted to cut bone during expansion from the collapsed delivery profile to the expanded deployed profile. The cutting surface may be sharp, jagged, etc. In some variations, the cutting surface is formed as an edge, having an angle of less than or about 90 degrees.

The bone anchor region may be made of a shape-memory material, including shape memory alloys (e.g., Nitinol, etc.), plastics, metals, ceramics, or the like. The bone anchor member may be pre-biased so that it self-expands from a closed to an open configuration. The open (or expanded) configuration may be referred to as an anchoring configuration, and the closed (or collapsed) configuration may be a delivery configuration. Tension may be applied (radial or tension across the struts) to keep the device in the delivery configuration.

A soft tissue attachment region is typically adapted to secure to soft tissue such as muscle, ligament, etc. (non-bone tissue). The soft tissue attachment region may include a soft tissue holdfast that secures to the tissue. Any appropriate soft tissue holdfast may be used. For example, the soft tissue attachment region may be adapted to receive a suture. The soft tissue holdfast may include or be made from a material through which a suture can be passed so that the soft tissue can be sewed to connect it to the soft tissue anchor region. For example, the soft tissue anchor may include a fabric, an elastomer, plastic, etc. In some variations that soft tissue holder is a mesh or framework onto which the soft tissue can be sutured. The soft tissue holdfast may include a clamping or gripping region for securing soft tissue between. For example, the soft tissue holder may include a cuff, a clamp, a press, or the like, for securing tissue. In some variations, the soft tissue holdfast is a combination of a suture and a frame, block, hook or loop.

Also described herein are soft tissue anchor device that include two sections that can be joined to form the soft tissue anchor. For example, described herein are soft tissue anchor devices comprising a bone anchor region and a soft tissue attachment region that interconnect. These two regions may be joined by a connector region that engages. The bone anchor region may include an elongate shaft, the shaft including a plurality of self-expanding struts extendable therefrom, the bone anchor region adapted to be positioned within a bone and having an expanded deployed profile and a collapsed delivery profile. The bone anchor region may also include a first connector region. The soft tissue attachment region may include a soft tissue holdfast configured to fixedly secure to soft tissue and a second connector region configured to mate with the first connector region and secure the soft tissue attachment region to the bone anchor region.

The connection between the bone anchor region and the soft tissue attachment region may be referred to as a connector region, and may include a first connector region and a second connector region that mate. The first and second connector regions may mate permanently or temporarily. For example, the first and second connector regions may be regions that secure together (e.g., they may be mutually threaded). The two regions may mate in any appropriate manner. In one variation, the first and second connector regions snap-fit together. The two regions may lock together.

The bone anchor region may be adapted to at least partially cut through the bone. These bone anchor regions may include one or more cutting surfaces. The cutting surface may be sharp, serrated, pointed, or the like. The cutting surface may be configured to cut through bone deeply or only partially. In general, the bone anchor is inserted in to a cavity that is pre-formed (or pre-existing) in bone, and allowed to expand; additional expansion may be applied. The bone may therefore be drilled or otherwise prepared. The soft tissue anchor devices described herein may be used with any type of bone, including cortical and spongy bone. The bone anchor may be adapted to cut through the bone as the struts of the bone anchor expand from the collapsed delivery profile to the expanded deployed profile. Thus, each self-expanding strut may include a cutting surface adapted to cut bone during expansion from the collapsed delivery profile to the expanded deployed profile.

The soft tissue attachment region of the two-part soft tissue anchor device typically includes a soft tissue holdfast that is configured to secure to soft tissue, as described above (e.g., the soft tissue holdfast may include a cuff, a hook, a loop, a clamping member, etc.). In addition, the soft tissue attachment region includes a second connector region that is configured to mate with the connector region on the bone anchor region.

In some variations the connector regions may include an additional connector element to which the first and second connectors connect. For example, a connector element may act as a spacer between the bone anchor region and the soft tissue anchor region. The connector may be flexible and/or adjustable. The connector may be stiff (rigid). In general the connector is a material such as a biocompatible polymer that can be safely implanted in tissue.

The two-part systems described herein may be beneficial because they may allow more flexibility in attaching the bone anchor region to the bone, and in attaching the soft tissue attachment region to the tissue.

Also described herein are systems for attaching soft tissue to bone. A system may include any of the soft tissue anchors described herein as well as an inserter for inserting at least the bone anchor region into the bone. For example, a system may include a soft tissue anchor device with a distal bone anchor region having an elongate shaft including a plurality of self-expanding struts extendable therefrom, the bone anchor region adapted to be positioned within a bone and having an expanded deployed profile and a collapsed delivery profile. The soft tissue anchor may also include a proximal a soft tissue holdfast configured to fixedly secure a soft tissue. The system may also include an inserter configured to releasably secure to the soft tissue anchor device and apply tension across the elongate shaft of the bone anchor region.

In general, an inserter attaches to one or more locations the devices, typically spanning the struts, so that they can apply force across the struts to prevent them from self-expanding until the bone anchor region has been delivered. In some variations the inserter includes a sleeve from which the bone anchor region is ejected (e.g., by a push rod). In some variations the inserter includes a rod that attaches to the distal end of the bone anchor region (releasably attaches) so that it can apply force to keep the bone anchor region in the delivery configuration. An inserter may also be configured (e.g., by releasably attaching to two points on the soft tissue anchor device) to apply additional force to expand the device, which may further help expand the device in the bone, even beyond the self-expansion.

Also described herein are methods of attaching soft tissue to bone. In general, these methods may include the steps of: delivering a bone anchoring region of a soft tissue anchor within a bone, the bone anchoring region having a plurality of self-expanding struts extendable therefrom, attaching the bone anchoring region to the bone by allowing the self-expanding struts to expand within the bone to anchor the soft tissue anchor therein; and securing a soft tissue to a soft tissue holdfast on the soft tissue anchor. The order of these steps may vary. For example, the tissue may be attached to the soft tissue holdfast prior to delivering or inserting the bone anchoring region into the bone and allowing it to self-expand and anchor.

The general method may be applied to both a one-piece soft tissue anchor device (in which the bone anchor region and the soft tissue attachment region are connected) and a two-piece or multi-piece soft tissue anchor device (in which the bone anchor region and the soft tissue attachment region are detached but connectable). Thus, in some variations, the method may include the step of securing the soft tissue holdfast to the bone anchoring region (e.g., connecting the soft tissue attachment region to the bone anchor region). The soft tissue attachment region may be attached either before, during or after the bone anchor region has been anchored to the bone (the securing step).

In some variations the step of delivering the bone anchor region may also include the step of further expanding the bone anchor region. For example, the bone anchor region may be further expanded by applying compressive force (as opposed to tension) across the shaft region including the struts. This additional force may help anchor the device in the bone.

The method may also include the step of visualizing the device within the bone before, during, or after the procedure.

### BRIEF DESCRIPTION OF THE DRAWINGS

FIG. 1 illustrates one variation of a soft tissue anchor device.

FIG. 2A is a side view of one variation of a soft tissue anchor device.

FIG. 2B is a perspective views of another variation of a soft tissue anchor device.

FIG. 3A is a side view of another variation of a soft-tissue device.

FIG. 3B is a perspective view of another variation of a soft-tissue device.

FIGS. 4A-4D show perspective (4A and 4B) and cross-sectional (4C and 4D) views of one variation of a bone anchor region that may be used with a soft tissue anchor device as described herein.

FIG. 5 is one variation of a bone anchor region attached to an inserter.

FIGS. 6A-6D illustrate the operation of an inserter and handle which may be used with at least the bone anchor region of a soft tissue anchor device.

FIGS. 7A-7J illustrate one method of attaching soft tissue to bone.

### DETAILED DESCRIPTION OF THE INVENTION

The soft tissue anchor devices, systems and methods described herein may aid in the attachment of ligaments, muscles, tendons, and other soft tissues to bone. Thus, the soft tissue anchor devices (“devices”) described herein may be used to treat pathologies or injuries. The devices, systems and methods described herein may be used in any appropriate body region, particularly bony regions. For example, the methods, devices and systems described herein may be used to connect soft tissue to the bones of the arms or legs.

In general, the soft tissue anchor devices described herein include a self-expanding bone anchor region including an elongate shaft that may be positioned within bone and expanded to anchor to the bone. The elongate shaft may include a plurality of self-expanding struts that expand to form a bow-shape. The soft-tissue anchoring device may also typically include one or more soft tissue attachment regions adapted to attach soft tissue thereto.

The soft tissue attachment regions may be connected to the bone anchor region, or they may be separate but connectable to the bone anchor region. Thus, a bone anchor region may be positioned in a bone (e.g., an arm bone) and a soft tissue attachment region may be connected to the bone anchor region. In some variations a connector region extends between the bone anchor region and the soft tissue attachment region. The connector region may be of a fixed length, or it may be adjustable; it may also be stiff, relatively stiff, or flexible. Soft tissue can be attached to the soft tissue anchor device before, during, or after the bone anchor region of the device is anchored to the bone, as will be described in more detail below.

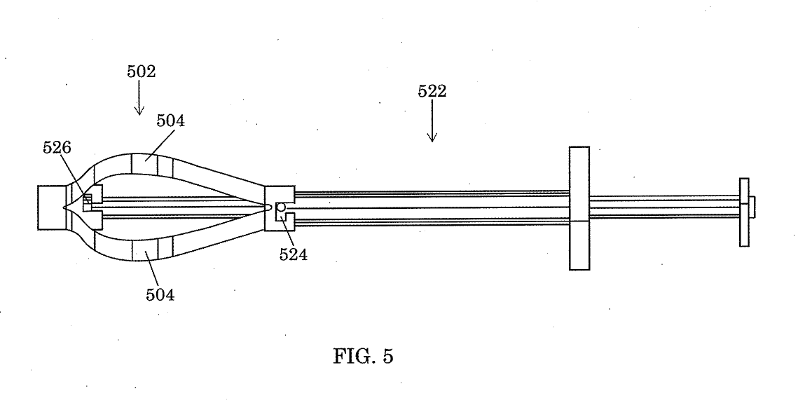

The bone anchor regions described herein may self-expand from a compressed profile having a relatively narrow diameter (e.g., a delivery configuration) into an expanded profile (e.g., a deployed configuration). The bone anchor region generally includes an elongate shaft forming a plurality of struts that may extend from the shaft body. The shaft is referred to as elongate since it extends linearly in the delivery configuration. The distal and proximal regions of the soft tissue anchor, and particularly the region surrounding the elongate shaft, may include one or more attachment regions configured to attach to an inserter for inserting (and/or removing) at least the bone anchor region of the soft tissue anchor, if not the entire soft tissue anchor. The inserter (also referred to as a delivery device) is generally configured to apply force to maintain the bone anchor region in the delivery conformation until it has been at inserted at least partially into the bone. FIGS. 5 and 6, described in detail below, illustrate variations of inserters.

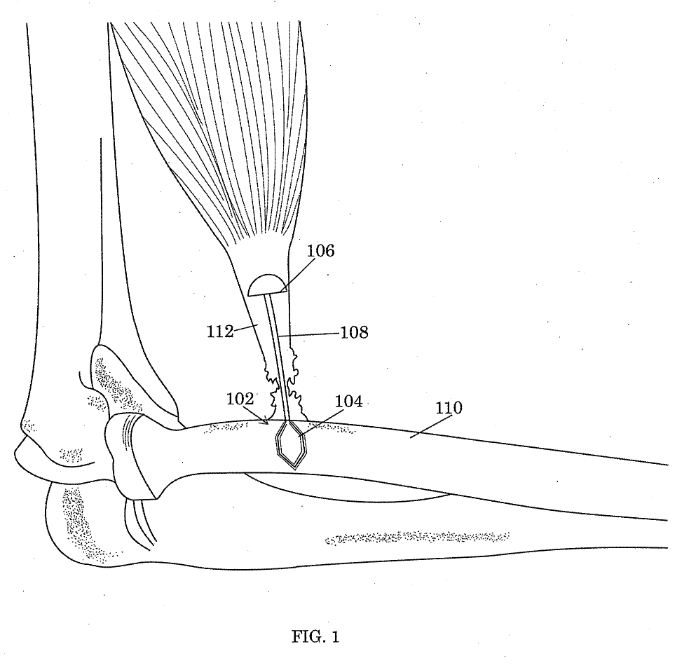

FIG. 1 illustrates one example of a soft tissue anchor device that is attaching soft tissue (tendon) to bone. In this example, the soft tissue anchor device 100 includes a bone anchor region 102 having a plurality of self-expanding struts 104 extendable therefrom. Device 100 further includes a soft tissue attachment region 106. This soft tissue attachment region 106 is attached to the bone anchor region 102 with a connector 108. In this example the connector 108 is two parallel wires extending from the bone anchor region 102 to the soft tissue anchor region 106. In other variations, the soft tissue anchor region is directly attached to the bone anchor region. Any appropriate connecting member can be used to connect the bone anchor region to the soft tissue attachment region. For example, a connector (also referred to as a connecting member or a connector region) may include pins, rods, screws, sutures, bendable or flexible materials, bio-absorbable materials, strings, wires, or other methods as known in the art.

In the example shown in FIG. 1, the soft tissue anchor region 106 is permanently attached to the bone anchor region 102. In some variations, these two regions are releasably attached.

As mentioned above, the bone anchor region 102 may include an elongate shaft that can self-expand from a collapsed delivery profile to an expanded deployed profile after insertion into bone 110, so that it can anchor the device within the bone. Further details of the elongate shaft and how bone anchor region 102 self-expands within bone will be described below.

The soft tissue attachment region 106 typically includes a soft tissue holdfast that secures to the particularly soft tissue of interest. In FIG. 1, the soft tissue holdfast is secured within the tendon and may be sutured in place. Any appropriate soft tissue holdfast may be used. For example the soft tissue holdfast may be a suturable material, including a fabric or polymeric material through which suture can pass, or a material (including metals and relatively stiff materials) having holes or passages for sutures. For example, a suture-penetrable biocompatible material (including bioabsorbable materials). In some variations, the soft tissue holdfast includes an adhesive (including tissue adhesives). In some variations, the soft tissue holdfast comprises a clamping or vise-like structure that secures the tissue between two or more surfaces (or within an annular surface) to apply pressure to retain the tissue. Combinations of these types of soft-tissue holdfast may be used. For example, a cuff formed of a suturable material may partially surround the tissue, and can be sutured through the material to further secure tissue within the cuff. In some variations, the soft tissue holdfast includes one or more sutures attached to it. The sutures can be passed through the tissue to secure it to the body of the soft tissue attachment region, and therefore to the rest of the soft tissue anchor device.

In other embodiments, soft tissue holdfast can comprise a hook, a loop, a wire, or the like. The soft tissue holdfast typically secures to soft tissue, either permanently, semi-permanently or temporarily.

As described above, the bone anchor region 102 can be inserted into bone 110 with a soft tissue attachment region 106 already connected either directly or via a connecting region or member 108. Soft tissue can be connected to the soft tissue attachment region either before or after engaging the bone anchor region in the bone. Alternatively, a soft tissue attachment region can be separate from the bone anchor region of the device. Tissue can then be secured to the soft tissue attachment region and the bone anchor region can be secured in the bone; thereafter (or before) the bone anchor region and the soft tissue attachment region can be connected, either directly or through a connector. The soft tissue attachment region and the bone anchor region can be attached either permanently or releasably.

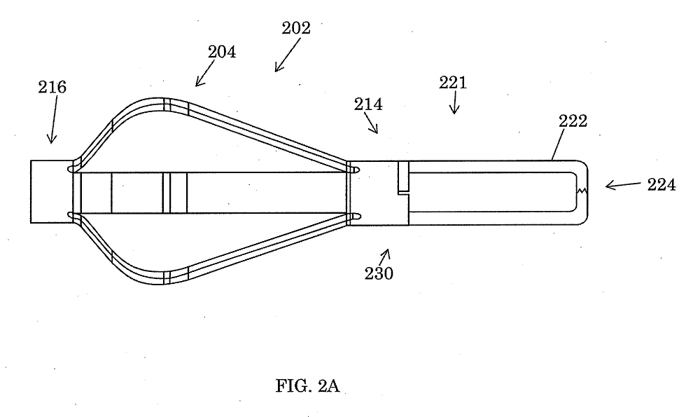

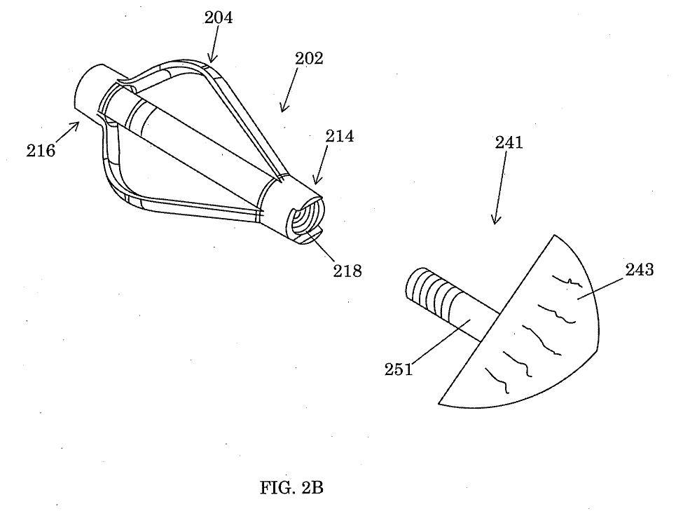

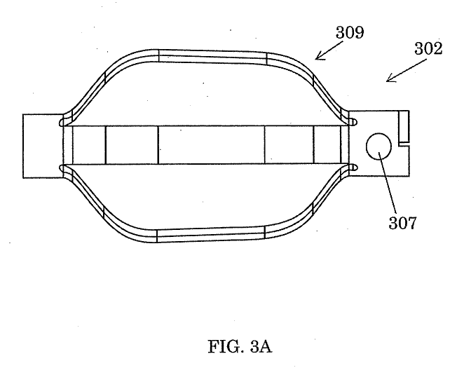

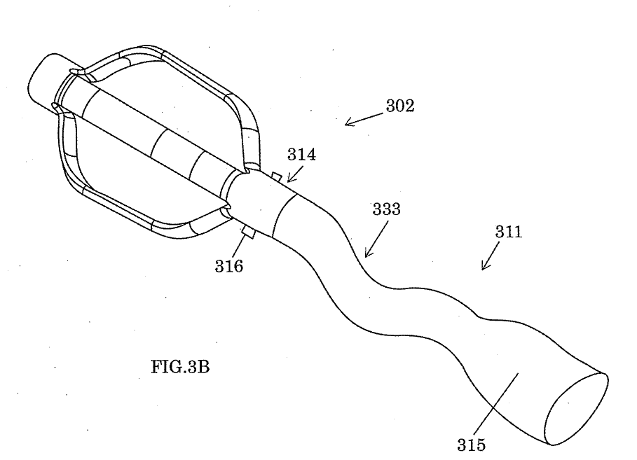

FIGS. 2A through 3B show exemplary soft tissue anchor devices including bone anchor regions and soft tissue attachment regions. Some examples also include connectors. For example, FIG. 2A shows a side view of one variation of a soft tissue anchor device including a bone anchor region 202 having struts 204 extending outwards from the shaft. Both FIGS. 2A and 2B show a bone anchor region 202 having an asymmetric expanded configuration. The bone anchor region 202 has an elongate shaft forming four struts 204, which may be formed by cutting four slots down the length of the shaft. The struts are pre-biased into the expanded configuration shown in the figures. In this example, the expandable bone anchor region 202 has a hollow central lumen, and a proximal end 214 and a distal end 216. By convention, the proximal end 214 is the end closest to the person inserting the device into a subject, and the distal end 216 is the end furthest away from the person inserting the device (towards the bone into which the device may be inserted).

The struts 204 of the elongate shaft 202 project axially outward, to form the asymmetric bow shape shown in FIGS. 2A and 2B (or the symmetric bow shown in FIGS. 3A and 3B). Three struts 204 are visible in each of FIGS. 2A and 2B. In general, each strut has a leading exterior surface. This leading surface may include a cutting surface adapted to cut bone. This cutting surface may be shaped to help cut through the bone (e.g., it may have a tapered region, or be sharp, jagged, serrated, etc.). The cutting surface (not apparent in FIGS. 2A and 2B) may extend along the entire length of the struts, or only over a portion of the struts. The cutting surface may be helpful in securing the device within the bone. For example, even if the bone (e.g., cortical bone) is not cut very deeply, the cutting edge may be sufficiently sharp to lodge the device in the bone enough to aid anchoring of the device.

The bone anchor region 202 is typically biased so that it is relaxed in the expanded or deployed configuration, as shown in FIGS. 2A and 2B. Force may be applied to the bone anchor region so that it assumes the narrower delivery profile. Thus, the struts 204 may elastically bend or flex from the extended configuration to the unextended configuration.

The struts 204 in these examples can include continuous curvature of bending struts. Continuous curvature of bending struts are struts that do not bend from the extended to an unextended configuration (closer to the central axis of the device shaft) at a localized point along the length of the shaft. Instead, the continuous curvature of bending struts are configured so that they translate between a delivery and a deployed configuration by bending over the length of the strut rather than by bending at a discrete portion (e.g., at a notch, hinge, channel, or the like). Bending typically occurs continuously over the length of the strut (e.g., continuously over the entire length of the strut, continuously over the majority of the length of the strut (e.g., between 100-90%, 100-80%, 100-70%, etc.), continuously over approximately half the length of the strut (e.g., between about 60-40%, approximately 50%, etc.). In some variations of the devices described herein, the struts do not have a continuous curvature of bending, but may be bent or hinged, or may include one or more notches along the length of the strut to facilitate bending.

The “curvature of bending” referred to by the continuous curvature of bending strut is the curvature of the change in configuration between the delivery and the deployed configuration. The actual curvature along the length of a continuous curvature of bending strut may vary (and may even have “sharp” changes in curvature). However, the change in the curvature of the strut between the delivery and the deployed configuration is continuous over a length of the strut, as described above, rather than transitioning at a hinge point. Struts that transition between delivery and deployed configurations in such a continuous manner may be stronger than hinged or notched struts, which may present a pivot point or localized region where more prone to structural failure.

The bone anchor region shown in FIG. 2A is connected directly to a soft tissue connector region 221, including a soft tissue holdfast 222. In this example, the soft tissue holdfast is a clamping mechanism 222 including two arms or jaws that project outward from the connector region 230. The jaws meet at the proximal end 224. In some variations the jaws or arms may be opened radially so that they can grasp tissue there between, and then shut to secure the tissue. In other variations the arms form a loop into which tissue can be passed and sutured or otherwise held in place.

FIG. 2B illustrates a similar example of a soft tissue anchor device in which the soft tissue attachment region 241 (including a soft tissue holdfast 243) is separable and can be connected to the bone anchor region 202. For example, in FIG. 2B the soft tissue attachment region 241 can be attached at the proximal end 214 of bone anchor region 202, which includes a first connector region 218 that is configured as a threaded receiving surfaced. As described in more detail below, this proximal end may also be adapted to releasably engage an inserter for inserting the elongate shaft into the bone.

In general, the bone anchor region is configured to secure to a soft tissue attachment region 241. As mentioned, in FIG. 2B the bone anchor region 202 includes a threaded connector 218 into which a connecting member and/or a soft tissue attachment region may connect. In FIG. 2B a connecting member 251 is connected to the soft tissue attachment region 241. The distal end of the connecting member 251 is threaded to allow it to be connected to the first connector region 218 on the bone anchor region. The connecting member and the soft tissue attachment region can also be attached at the distal end of the bone anchor region.

In this example, the connecting member extends the distance between the soft tissue attachment region and the bone anchor region. The connecting member 251 shown is a rigid rod, however, any other appropriate connecting member may be used. For example, the connecting member may be flexible or semi-flexible (e.g., flexible in parts, etc.). In some variations the connecting member referred to as a connecting region. The connecting member may be a tether. In some variations the connecting member is adjustable. For example, the connecting member may be a telescoping member that can change length (and may be lockable at a desired length, e.g., via a set screw, etc.) In some variations.

In FIG. 2B the soft tissue attachment region includes a soft tissue holdfast that comprises a flexible material 243 through which a suture may pass. In operation a soft tissue (e.g., tendon, muscle, etc.) maybe placed in or against the material 243 and sutured to or between the material to hold it in place. Thus, in this variation the soft tissue holdfast acts as a base against which the soft tissue maybe sutured to hold it in place. In some variations the soft tissue holdfast may also include a tissue adhesive (e.g., a biocompatible adhesive).

The distal region 216 of the device (adjacent to the distal end of the bone anchoring region in FIGS. 2A and 2B) may also be adapted to attach to an inserter.

In some variations, the soft tissue attachment region may be permanently connected to the bone anchoring region. For example, opening 218 may be formed of, or may include, a material that is crushable (e.g., a porous, frangible, or compressible material) that is configured to be compressed by the connecting member and soft tissue attachment region as or after it is inserted. This may lock the two regions together.

In another variation, the first connector region includes a post or projection to which the connecting member or a second connector region on the soft tissue attachment region mates. For example, the opening 218 may include a post projecting from an outer (or inner) surface of the over which the connecting member or second connector region slides.

As mentioned, any appropriate connection between the bone anchor region and the soft tissue attachment region (including any connector members) may be used. For example, the connection may be a friction fitting. In some variations the connection is a snap fitting.

The soft tissue anchor devices described herein generally have two or more releasable inserter attachment regions for attaching to an inserter. For example, a soft tissue anchor device may include at a first attachment region for an inserter at the proximal end of the bone anchor region and a second attachment region for an inserter at the distal end of the bone attachment region. Generally the attachment regions for the inserter spans bone anchor region, so that the inserter may apply force to prevent the bone anchor region from expanding until it has been inserted and/or positioned. For example, the inserter may apply force across the elongate shaft of the bone anchor region (e.g., to apply tension to the struts and keep them in the contracted or delivery configuration). In some variations the soft tissue anchor device includes only a single attachment region (e.g., proximal to the bone anchor region). In this variation, the device includes a seating region distal to the bone anchor region against which a portion of an inserter (e.g., a rod) can press to apply force to control the configuration of the bone anchor region. In some variations of the self-expanding bone anchor region, the force to alter the configuration of the device from the delivery to the deployed configuration comes from the material of the elongate shaft itself (e.g., from a shape-memory material), and thus only a single attachment region (or one or more attachment region at a single end of the elongate shaft) is necessary.

FIGS. 3A and 3B illustrate another variation of a soft tissue anchor device. In this variation the bone anchor region is symmetric. FIG. 3A shows the distal end of the soft tissue anchor device (including the bone anchor region 309 and a first connector 307) without the soft tissue attachment region connected. FIG. 3B shows the soft tissue anchor with the soft tissue attachment region 311 connected. In this example, the connector between the bone anchor region and the soft tissue attachment region (including the soft tissue holdfast) is a snap-fit connector 314 having a male portion on the proximal side and a female portion 307 on the distal side. The male portion includes a tab (or tabs) 316 that can be inserted into the connector. The tab may flex as it is inserted and may “pop” back out into the holes 307 of the connector to lock into place. In some variations the tab is a ramp or hook region; the ramped surface allows it to be inserted, but makes it difficult to withdraw.

In FIG. 3B, the soft tissue anchor includes a flexible connector region 333 between the bone anchor region and the soft tissue holdfast 315. In this example, the connector region includes the male connector 316 that attaches to the first connector 307 at the proximal end of the bone anchor region. The connector region shown is formed of a flexible material (e.g., a woven or extruded material) and maybe made of the same material as the soft tissue anchor. The connector may be a flat strip or a “tube” (including a hollow or non-hollow tube), as shown in FIG. 3B.

The soft tissue soft tissue holdfast of FIG. 3B is a cuff 315, which may partially surround tissue and through which sutures may be applied. Suturing soft tissue to the cuff material may help distribute the force from the suture across a larger area of the soft tissue. The material forming the soft tissue holdfast is typically biocompatible and may also include therapeutic materials such as growth-promoting materials, antibiotic materials, anticoagulants, etc.

In general, the soft tissue anchor devices may include one or more therapeutic materials in one or more regions. For example, bone growth-promoting materials (e.g., BMPs, etc.) or other in-growth promoting materials may be included as a coating or impregnation in the bone anchor region. As described in greater detail below, the bone anchor region may be cemented or otherwise affixed to the bone; therapeutic materials may also be include as part of the flowable material added to secure the device in the bone.

Although the bone anchor regions shown in FIGS. 2A to 3B are shown in their expanded (deployed or anchoring) states, they may be held in an elongate delivery state by applying force, as described above. Furthermore, although these examples have a roughly cylindrical profile (in the delivery state in particular), other cross-sectional shapes may be preferred. For example, it may be beneficial to have devices with triangular, rectangular (including square) and other profiles, particularly flat profiles. The edges of these devices may be incorporated to form a cutting edge in the expandable strut. Furthermore, the bone anchor region of the soft tissue anchor device may have a different cross-sectional profile along the length of the device, which may result in struts having different edge portions and/or diameters that vary along the length, and may help with insertion (or removal) of devices into the bone.

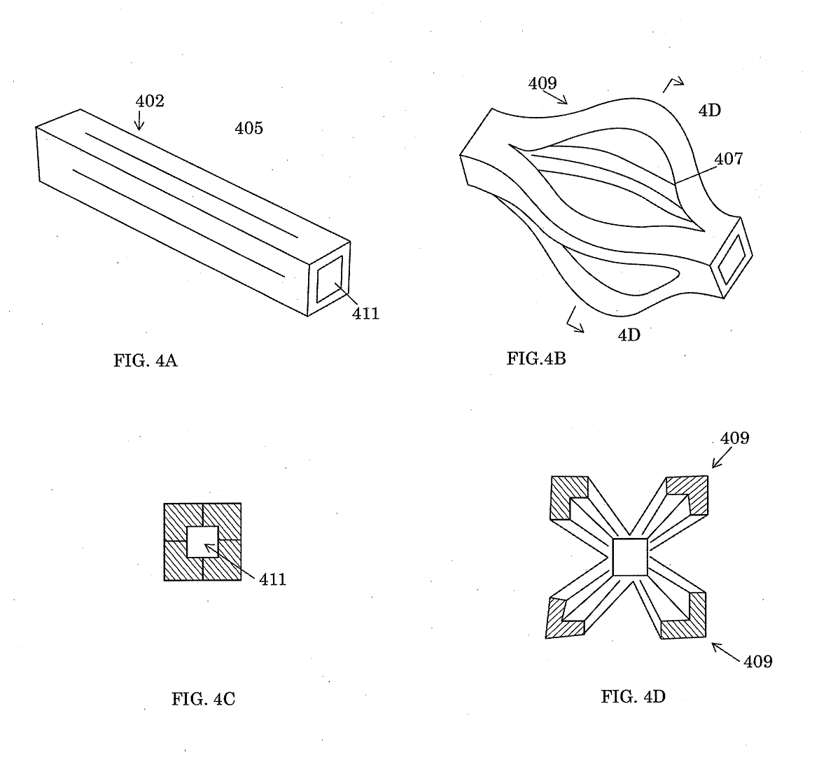

FIGS. 4A to 4D illustrate one variation of a bone anchor region 402 that forms struts with sharp cutting edges that may help the device insert into the bone and secure (anchor) it in place. FIG. 4A shows a perspective view of just the bone anchor region of a soft tissue anchor device. The other features and elements of a soft tissue anchor device (e.g., attachment and connector regions, and the soft tissue attachment region) have been removed to illustrate just features of the bone anchor region. It should be understood that this example of a bone anchor region may be used with any of the devices described herein including them features not illustrated in FIGS. 4A-4D.

FIG. 4A shows a bone anchor region in the unexpanded or delivery configuration, and FIG. 4B shows the same bone anchor region in an expanded configuration. As mentioned, the bone anchor region may be pre-biased in the expanded configuration. For example, the bone anchor region may be formed at least partially of a shape memory material that is configured so that the relaxed state at body temperature is the expanded state. Force may be applied to hold the device in the linear, delivery configuration, shown in FIG. 4A. For example, force may be applied radially (e.g., within a sleeve or cannula) or by applying tension (e.g., pulling on either end of the bone anchor region. The bone anchor region of FIG. 4A is formed as a four-sided (rectangular or square) cylinder having four silts cut partially along the length to form the four struts 407. The struts 407 each include a cutting edge region 409 formed by the corner of the rectangular cylinder when the struts expand. The inner diameter of the bone anchor region shown is hollow 411, which may allow for the delivery of a flowable material including a cement or other material, as described below.

FIGS. 4C and 4D show cross-sections through the bone anchor region. For example, FIG. 4A shows a cross-section through the middle of the delivery configuration shown in FIG. 4A. FIG. 4D shows a cross section through the middle region of the expanded configuration shown in FIG. 4B, as indicated by the arrows 4D. In FIG. 4D, the cutting edge 409 of the struts is apparent. In this example, the cutting edge is approximately a 90 degree angle edge. In other variations the edge may be made sharper by increasing the curvature of the edge region.

The bone anchor region embodiment shown in FIGS. 4A-4D illustrates one variation of a bone anchor region including an elongate shaft 402 forming struts 407 that may cut through bone during expansion from a collapsed delivery profile to an expanded deployed profile. Edge region 409 provides a sharp or cutting surface on the self-expanding struts 404 that allows the elongate shaft to apply a bone cutting force to the bone during expansion from the collapsed delivery profile to the expanded deployed profile.

Other variations of bone anchor regions may also include cutting edges. For example, a bone anchor region with an elongate shaft having a cylindrical or round cross-section (e.g., FIGS. 2A-3B) can have an additional edge or protrusion extending from the self-expanding struts to provide a cutting force to bone during expansion.

The dimensions of the struts may be adjusted to calibrate or enhance the strength of the elongate shaft, and/or the force that the elongate shaft exerts when self-expanding. For example, thicker struts (e.g., thicker cross-sectional area) may exert more force when self-expanding than thinner struts. This force may also be related to the material properties of the struts.

The struts may be made of any appropriate material. In some variations, the struts and other regions of the soft tissue anchor device are made of substantially the same material. Different portions of the bone anchor region (including the struts) may be made of different materials. In some variations, the struts may be made of different materials (e.g., they may be formed of layers, and/or of adjacent regions of different materials, which may have different material properties). The struts may be formed of a biocompatible material or materials. It may be beneficial to form struts of a material having a sufficient spring constant so that the device may be elastically deformed from the deployed configuration into the delivery configuration, allowing the elongate shaft to self-expand back to approximately the same deployed configuration. In some variation, the strut is formed of a shape memory material that may be reversibly and predictably converted between the deployed and delivery configurations. Thus, a list of exemplary materials may include (but is not limited to): biocompatible metals, biocompatible polymers, polymers, and other materials known in the orthopedic arts. Biocompatible metals may include cobalt chromium steel, surgical steel, titanium, titanium alloys (such as the nickel titanium alloy Nitinol™), tantalum, tantalum alloys, aluminum, etc. Any appropriate shape memory material, including shape memory alloys such as Nitinol™ may also be used.

Other portions of the soft tissue anchor device may be made of the same material(s) as the struts, or they may be made of a different material. Any appropriate material (preferably a biocompatible material) may be used, including any of those materials previously mentioned, such as metals, plastics, ceramics, or combinations thereof. In some variations, portions of the soft tissue anchor device can also be formed from suitable polymers include polyesters, aromatic esters such as polyalkylene terephthalates, polyamides, polyalkenes, poly(vinyl)fluoride, PTFE, polyarylethyl ketone, and other materials. Various alternative embodiments of the soft tissue anchor device and/or components could comprise a flexible polymer section (such as a biocompatible polymer) that is rigidly or semi-rigidly fixed.

As mentioned briefly above, a bone anchor region (including struts), may also include one or more coating or other surface treatment (embedding, etc.). Coatings may be protective coatings (e.g., of a biocompatible material such as a metal, plastic, ceramic, or the like), or they may be a bioactive coating (e.g., a drug, hormone, enzyme, or the like), or a combination thereof. For example, the bone anchor region may elute a bioactive substance to promote or inhibit bone growth, vascularization, etc. In one variation, the device includes an elutible reservoir of bone morphogenic protein (BMP).

As previously mentioned, the bone anchor region may be formed about a central elongate hollow body. In some variations, the struts are formed by cutting a plurality of slits long the length (distal to proximal) of the elongate body. This construction may provide one method of fabricating the bone anchor region, however the bone anchor regions described herein are not limited to this construction. If formed in this fashion, the slits may be cut (e.g., by drilling, laser cutting, etc.) and the bone anchor region may be pre-set or pre-biased into a deployed shape so that this configuration is the default, or relaxed, configuration in the body. For example, the struts may be formed by plastically deforming the material of the struts into the deployed configuration. In general, any of the bone anchor regions may be thermally treated (e.g., annealed) so that they retain this deployed configuration when relaxed. Thermal treatment may be particularly helpful when forming a strut from a shape memory material such as a nickel-titanium alloy (e.g., Nitinol™) into the deployed configuration.

Any of the bone anchor regions described herein may be used with an inserter or applicator that can be used to position the self-expanding bone anchor region of the device within the subject's bone. An applicator or inserter may be used to insert just the portion of the soft tissue anchor device including the bone anchor region, or it may be used to insert the entire soft tissue anchor device (including an attached soft tissue attachment region). In some variations a separate soft tissue attachment region inserter or applicator is used to secure the soft tissue holdfast to a soft tissue, and/or to connect the soft tissue attachment region to the bone anchor region of the device.

FIG. 5 shows a partial view of one variation of a soft tissue anchor device including a bone anchor region 502 having a plurality of self-expanding struts 504. The device is removably attached to an inserter 522. In this example, an inserter attachment region 524 at the proximal portion of the bone anchor region is configured as an L-shaped notch, as is the attachment region 526 at the distal portion of the device. Other variations of the may include different inserter attachment regions that allow the soft tissue anchor device to be releasably connected to the inserter. For example, the inserter attachment region may include a threaded region, a magnetic region, a clamping region, or the like. The inserter attachment region at the proximal end of the bone anchor device may also be configured as the connector region for connecting to the soft tissue attachment region, as mentioned above. In soft tissue anchor device having two (or more) inserter attachment regions, the attachment regions may be configured differently. In some variations the attachment region is just a surface against which a portion of the inserter abuts.

In general, an inserter includes an elongate body having a distal end to which the soft tissue attachment device may be attached and a proximal end which may include a handle or other manipulator that coordinates converting an attached elongate shaft from a delivery and a deployed configuration, and may also allow a user to selectively release the elongate shaft from the distal end of the inserter.

FIGS. 6A-6D illustrate the proximal end of an inserter connected to a handle that can controllably actuate the inserter. In this example, the bone anchor region, shown in FIGS. 6A and 6C, is in the deployed configuration (FIG. 6A) when the handle is “open,” as shown in FIG. 6B. By squeezing the handle (e.g., by rotating the finger grip region towards the palm region), as shown in FIG. 6D, the inserter can apply force between the proximal and distal portions of the bone anchor region. The tension can put the bone anchor region of the device in a delivery configuration, as shown in FIG. 6C. The inserter may be secured to lock the bone anchor region in this configuration until it has been inserted.

Inserter may also be referred to as applicators. In general, inserting a structure into bone and soft tissue unaided takes a great deal of skill, as these parts of the body typically small and may be difficult to access. The devices and method described herein may be used in conjunction with any appropriate visualization technique, including 3-D imaging, to place the devices into the bone and soft tissue.

### Methods of Attaching Soft Tissue to Bone

As mentioned above, any of the soft tissue anchor devices described herein may be used to repair a bone. A method of treating a bone using the soft tissue anchor devices described herein typically involves attaching soft tissue to the soft tissue attachment region of the device, and anchoring the bone anchor region of the device in a cavity or opening in a bone by expanding (e.g., self-expanding) the device in the cavity. The order of these steps may be different. For example, the soft tissue anchor device may be attached to the bone first, before the soft tissue is attached to the soft tissue holdfast of the soft tissue attachment device. In addition, the method may also include additional steps. For example, in some variations, the soft tissue attachment region is a separate but connectable component of the soft tissue anchor device that is attached to the bone anchor region of the device either before, during, or after the bone anchor region has been secured to the bone. Other additional steps are described below.

The general step of securing the bone anchor region of the device to the bone in a cavity of the bone may include the step of forming the cavity in the bone. For example, the bone may be drilled. The drilled hole or cavity is typically large enough to fit at least a portion of the length of the bone anchor region of the device into the bone. The width is typically large enough to accommodate the width of the distal end of the soft tissue device, including the bone anchor region in the collapsed or delivery configuration; the width is typically smaller than the expanded diameter of the bone anchor region. In some variations the bone anchor region is configured to expand between about 1.5 to 5 times the diameter of the collapsed (delivery) configuration. The bone opening into which the device is inserted may also be a naturally occurring or pre-existing bone opening, depending upon the intended application.

When securing the bone anchor region of the device to the bone, the bone anchor region is typically held in the contracted position using an inserter, which may then be used to position the device in the bone cavity. Once the position is achieved, the bone anchor region may be released (all at once, or gradually) from the device so that it can self-expand within the cavity. The struts may therefore bow outward and contact the walls of the cavity. Depending on the bone anchor region, the type of bone, the sizes of the device and the bone cavity, the struts may cut through the bone (at least partially), thereby further anchoring it. After allowing the bone anchor region to self-expand, additional force may optionally be applied to further expand the device. For example, the inserter may apply compressive force over the bone anchor region to further expand it. The inserter may be removed from the soft tissue anchor device. For example, the attachment sites between the inserter and the soft tissue anchor device (typically at either end of the bone anchor region) may be disengaged by unscrewing or unlocking, e.g., by rotating, by activating a push-button, etc. In some variations the soft tissue anchor device may be repositioned by re-engaging the inserter with the soft tissue anchor device and applying tension across the bone anchor region to collapse it again so that it can be repositioned.

Once the soft tissue anchor device is positioned in the bone, it may be further secured in place by the addition of a material (e.g., bone cement or other fluent material) or securing screw, pin, etc. If a bone cement is used, any appropriate cement may be used, including flowable materials, such as a bone filling composition or cement, which may include biological materials, synthetic materials, inorganic materials, or bioactive agents (or any combinations thereof). Other bone filling compositions or cement include PMMA (polymethylmethacrylate) which may be injected into the cavity into which the soft tissue anchor has been positioned. In particular, the central passageway through the soft tissue anchor device (e.g., particularly in the bone anchor region) may be used to deliver a flowable material. For example, material may be delivered through a trocar and cannula into the passageway of a device.

There are many suitable materials known in the art for filling in vacant spaces in bone and which may be used herein. Some of these materials or compositions are biological in origin and some are synthetic, as described in U.S. patent application Ser. No. 11/468,759, which is incorporated by reference herein. The material may be applied to flow into the open space within the bone anchor region and to some degree, into the peripheral area surrounding the device. The device (e.g., the bone anchor region passageway) may be capped or blocked to prevent excess loss of material applied, and help confine it somewhat to the bone. The process of applying flowable material may also be observed to control the amount and location applied. For example, a flowable cementing material may contain radiopaque material so that when injected under live fluoroscopy, cement localization and leakage can be observed.

Another example of bone cementing material is a ceramic composition including calcium sulfate calcium hydroxyapatite, such as Cerament™, as manufactured by BoneSupport AB (Lund, Sweden). Ceramic compositions provide a dynamic space for bone in-growth in that over time, they resorb or partially resorb, and as a consequence provide space for in-growth of new bone. Bioactive agents may also be included in a cementing composition, such as osteogenic or osteoinductive peptides, as well as hormones such at parathyroid hormone (PTH). Bone Morphogenetic Proteins (BMPs) are a prominent example of effective osteoinductive agents, and accordingly, a protein such as recombinant human BMP-2 (rhBMP-2) may included in an injected bone-filling composition. In this particular context, BMPs promote growth of new bone into the regions in the interior of the expanded struts and around the periphery of the device in general, to stabilize the device within new bone. A more fundamental benefit provided by the new bone growth, aside from the anchoring of the device, is simply the development of new bone which itself promotes healing. In some variations, antibiotics may be included. In general, any appropriate flowable material may be injected into the bone cavity or the device, particularly the passageway formed through the bone anchor region. Some variations of the devices described herein include a passageway for a flowable material through the entire length of the device. In some variations having a separable or connectable soft tissue attachment region, the pathway may be caped or blocked once the connectable soft tissue attachment region is connected.

Positioning and securing the soft tissue anchor device to the bone as described above may be done either before or after the soft tissue has been attached to the soft tissue holdfast region of the device.

Soft tissue is typically attached to the soft tissue holdfast in the soft tissue attachment region. Attachment may be by adhesive, clamping, suturing, or other appropriate means, or combinations. For example, in some variations the soft tissue holdfast is a surface through which sutures can be passed or tied so that the soft tissue can be sutured to the soft tissue holdfast.

In variations in which the soft tissue attachment region of the device is not initially connected to the bone anchor region, the soft tissue attachment region may be attached to the bone anchor region after soft tissue has been secured to the soft tissue holdfast. This may involve direct attachment of the distal end of the soft tissue attachment region to the proximal end of the bone anchoring region, or the use of a connector member to expend the spacing between the two regions. A connector member may be located at the distal end of the soft tissue anchor region. In some variations the connector member is a tether that is flexible. The connector member may link to the bone anchor region of the soft tissue anchor device.

The attachment of the soft tissue anchoring region to the bone anchor region forms the complete soft tissue anchor device, and may be a permanent attachment, as mentioned above. A soft-tissue attachment region inserter or applicator may be used to attach this region of the device to the bone anchor region. Thus, the soft tissue attachment region may include a releasable connector configured to connect the soft tissue attachment region to an inserter.

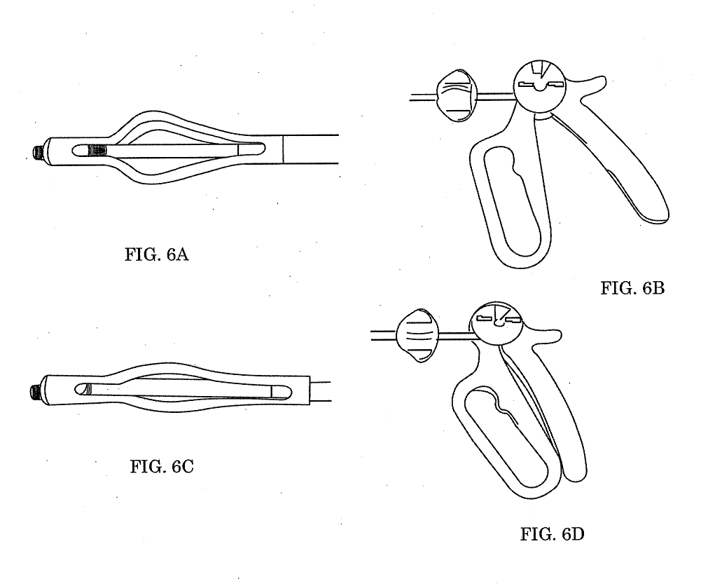

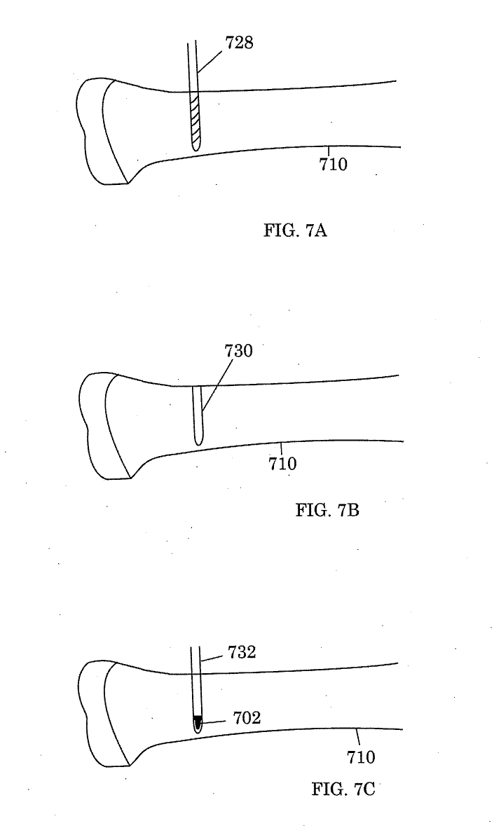

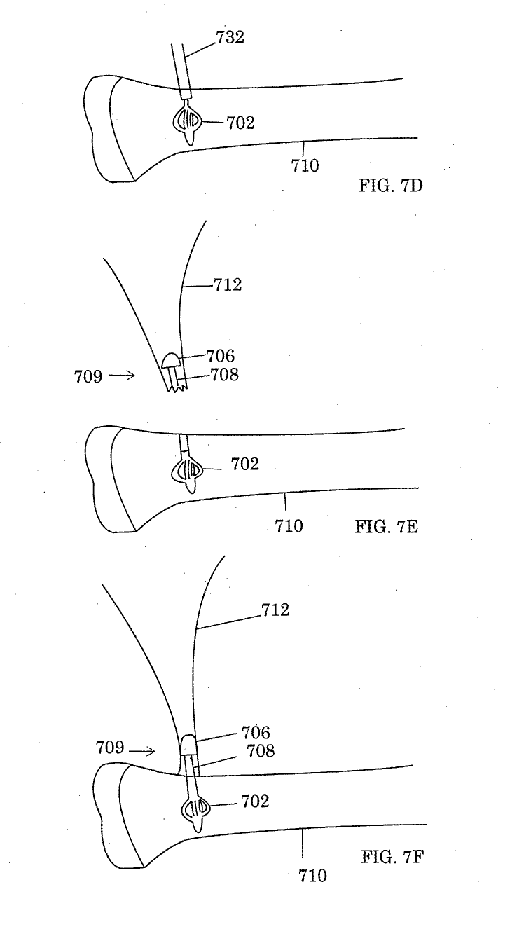

FIGS. 7A-7J illustrate one variation of a method using a system and device as described herein. In this example, a soft tissue anchor device is used to reattach a tendon to a bone. FIG. 7A illustrates using a drill 728 to form a cavity or passageway within the bone 710. An elongate instrument is used to drill the hole, and the width and depth may be matched to the soft tissue anchor device to be used, or the soft tissue anchor device can be matched to the size of the cavity formed. The location of the cavity formed in the bone may be done after determining an appropriate soft tissue attachment point so as to reattach the soft tissue in an appropriate position, such as a position that best approximates the natural position. Such alignment may be attained by methods well known in the art. The passageway can be formed by any appropriate method and is not limited to drilling. For example, a cavity may be found or enlarged by hammering, chiseling, reaming, etc.

FIG. 7B shows the formed passageway 730 prepared to receive a soft tissue anchor device. FIG. 7C shows the distal end of one variation of an inserter/delivery device 732 within the cavity formed. In this example the inserter is a cannula device having an outer sleeve into which the distal bone anchoring region of the soft tissue anchor device is held. The cannula applies radial force to keep the bone anchoring region compressed. A push rod in the cannula may be used to eject the bone anchoring region of the soft tissue anchor device. In some variation this push rod is releasably connected to the soft tissue anchor device, which may allow the position of the device to be adjusted (e.g., by pulling the device back into the cannula).

In FIG. 7C the distal end of the soft tissue anchor device is just beginning to exit the cannula sleeve that radially envelopes the bone anchoring region of the device 702 being inserted. FIG. 7D shows the bone anchoring region 702 after expansion of the self-expanding struts once the majority of the elongate shaft of the bone anchoring region has been pushed out of the distal end of the cannula 732. The cannula has also been partially withdrawn from the implant site as the device is inserted.

A cement or other flowable material may also be applied to fill the bone region around the soft tissue anchor device shown in FIG. 7D. For example, the cannula of the inserter may be used to apply a flowable material into the central passageway of the bone anchor region, as previously described. A bone cement or filler may further help secure the soft tissue anchor device in the bone.

In this example the soft tissue attachment region 709 is a connectable portion of the device that is separately attached to the appropriate soft tissue. In FIG. 7E the soft tissue attachment region 709 has been attached to a tendon 712 at the soft tissue holdfast 706. The soft tissue holdfast 706 shown is generic, and may include staple, sutures, adhesives, or the like, to secure the tendon. For example, as described above, the soft tissue attachment region may be a hook, a loop, or holes formed in or on the elongate shaft which can be attached to the soft tissue, such as by suturing. The soft tissue attachment region includes a connecting member 708 which connects to the proximal end of the bone anchor region 702 as shown in FIG. 7F.

In this example, the soft tissue attachment region 709 is connected to the bone anchor region 702 after the soft tissue holdfast 706 has been attached to the soft tissue 712. In an alternative embodiment, the soft tissue attachment region 709, including connecting member 708, is secured to bone anchor region 702 before attaching the soft tissue to soft tissue holdfast.

As shown in FIG. 7F, the soft tissue attachment region 706 and the bone anchor region 702 have been joined so that the tendon 712 is secured to the bone.

In some variations, the soft tissue anchor device can be implanted as a single integrated device. When the device is a single integrated device, the soft tissue can be attached to soft tissue holdfast (and thus the soft tissue attachment region) before implanting the elongate shaft in bone, or the elongate shaft can be implanted in bone before attaching the soft tissue to soft tissue attachment region. The delivery device or inserter used to insert the bone anchor region of the device may therefore be adapted for use with devices in which the soft tissue attachment region is linked to the bone anchor region.

While the devices, systems, and methods for using them have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

We claim:

1. A soft tissue anchor device comprising:
a distal bone anchor region having an elongate shaft including a plurality of self-expanding struts extendable therefrom, the bone anchor region adapted to be positioned within a bone and having an expanded deployed profile and a collapsed delivery profile;
a proximal soft tissue holdfast configured to fixedly secure a soft tissue.

2. The device of claim 1 further comprising a connecting region between the elongate shaft and the tissue attachment region.

3. The device of claim 2, wherein the connecting region comprises a flexible connecting region.

4. The device of claim 2, wherein the connecting region comprises an adjustable connecting region.

5. The device of claim 1, wherein the bone anchor region is adapted to cut through the bone during expansion from the collapsed delivery profile to the expanded deployed profile.

6. The device of claim 1, wherein each self-expanding strut includes a cutting surface adapted to cut bone during expansion from the collapsed delivery profile to the expanded deployed profile.

7. The device of claim 1, wherein the bone anchor region comprises a shape memory material.

8. The device of claim 1, wherein the soft tissue holdfast is adapted to receive a suture.

9. The device of claim 1, wherein the soft tissue holdfast comprises a cuff.

10. The device of claim 1, wherein the soft tissue holdfast comprises a hook.

11. The device of claim 1, wherein the soft tissue holdfast comprises a loop.

12. The device of claim 1, wherein the soft tissue holdfast comprises a clamping member.

13. A soft tissue anchor device comprising:
a bone anchor region comprising:
an elongate shaft, the shaft including a plurality of self-expanding struts extendable therefrom, the bone anchor region adapted to be positioned within a bone and having an expanded deployed profile and a collapsed delivery profile; and
a soft tissue attachment region comprising:
a soft tissue holdfast configured to fixedly secure to soft tissue;
wherein the soft tissue attachment region is configured to be connected at the proximal end of the bone anchor region.

14. The device of claim 13, further comprising a first connector region at the proximal end of the bone anchor region and a second connector region at the distal end of the soft tissue attachment region.

15. The device of claim 13, wherein the first connector region is configured to snap-fit with the second connector region.

16. The device of claim 13, wherein the first connector region is configured to lock to the second connector region.

17. The device of claim 13, wherein the bone anchor region is adapted to cut through the bone during expansion from the collapsed delivery profile to the expanded deployed profile.

18. The device of claim 13, wherein each self-expanding strut includes a cutting surface adapted to cut bone during expansion from the collapsed delivery profile to the expanded deployed profile.

19. The device of claim 13, wherein the elongate shaft comprises a shape memory material.

20. The device of claim 13, wherein the soft tissue holdfast is adapted to receive a suture.

21. The device of claim 13, wherein the soft tissue holdfast comprises a cuff.

22. The device of claim 13, wherein the soft tissue holdfast comprises a hook.

23. The device of claim 13, wherein the soft tissue holdfast comprises a loop.

24. The device of claim 13, wherein the soft tissue holdfast comprises a clamping member.

25. A system for attaching soft tissue to bone, the system comprising:
a soft tissue anchor device comprising:
a distal bone anchor region having an elongate shaft including a plurality of self-expanding struts extendable therefrom, the bone anchor region adapted to be positioned within a bone and having an expanded deployed profile and a collapsed delivery profile; and
a proximal a soft tissue holdfast configured to fixedly secure a soft tissue; and
an inserter configured to releasably secure to the soft tissue anchor device and apply tension across the elongate shaft of the bone anchor region.

26. The system of claim 25, wherein the bone anchor region is adapted to cut through the bone during expansion from the collapsed delivery profile to the expanded deployed profile.

27. The device of claim 25, wherein the struts include a cutting surface adapted to cut bone during expansion from the collapsed delivery profile to the expanded deployed profile.

28. A method of attaching soft tissue to bone comprising the steps of:
delivering a bone anchoring region of a soft tissue anchor within a bone, the bone anchoring region having a plurality of self-expanding struts extendable therefrom;
attaching the bone anchoring region to the bone by allowing the self-expanding struts to expand within the bone to anchor the soft tissue anchor therein; and
securing a soft tissue to a soft tissue holdfast on the soft tissue anchor.

29. The method of claim 28 further comprising securing the soft tissue holdfast to the bone anchoring region.

30. The method of claim 28, wherein the securing step is performed before the attaching step.

31. The method of claim 28 further comprising visualizing the device within the bone.

32. The method of claim 28 further comprising drilling a hole into the bone through which the bone anchoring region may be inserted.

33. The method of claim 28 further comprising applying force to further expand the elongate shaft of the bone anchoring region within the bone.

34. The method of claim 28 further comprising applying a flowable material in or around the bone anchoring region of the soft tissue anchor.

35. The method of claim 34, wherein the flowable material comprises a biologic or synthetic material to promote anchoring and to allow for new bone in growth.
